(19) United States
(12) Patent Application Publication
(10) Pub. No.: US 2025/0312304 A1
(43) Pub. Date: Oct. 9, 2025

(54) RETINOL ACID PATHWAY MODULATORS FOR TREATMENT OF CONGENITAL VASCULAR MALFORMATION

(71) Applicants: University of South Carolina, Columbia, SC (US); The Regents of the University of California, Oakland, CA (US)

(72) Inventors: Wenbin Tan, Columbia, SC (US); Vi Nguyen, Columbia, SC (US); J. Stuart Nelson, Irvine, CA (US)

(73) Assignees: University of South Carolina, Columbia, SC (US); The Regents of the University of California, Oakland, CA (US)

(21) Appl. No.: 19/044,693

(22) Filed: Feb. 4, 2025

Related U.S. Application Data
(60) Provisional application No. 63/574,974, filed on Apr. 5, 2024.

(51) Int. Cl. A61K 31/203; A61K 31/4184; A61K 31/4436; A61N 5/067

(52) U.S. Cl. CPC A61K 31/203; A61K 31/4184; A61K 31/4436; A61N 5/067

(57) ABSTRACT

Described herein are systems and methods for repurposing of a category of FDA-approved drugs in combination with laser therapy for treatment of blood vessel abnormalities to improve the current efficacy of laser therapy.

### STATEMENT REGARDING FEDERALLY SPONSORED RESEARCH

This invention was made with government support under R01AR073172 from NIH/NIAMS and HT94252310008 from DoD. The government has certain rights in the invention.

### TECHNICAL FIELD

The subject matter disclosed herein is generally directed to systems, methods, and treatment regiments for repurposing of a category of FDA-approved drugs in combination with laser therapy for treatment of blood vessel abnormalities to improve the current efficacy of laser therapy.

### BACKGROUND

Congenital vascular malformations (CVM) are a major threat to public health with a 1.5% incident rate in the general population. Congenital capillary malformation (CM), a.k.a., port-wine birthmarks or stains (PWB or PWS), is the most common types of CVM with an estimated prevalence of 0.3-0.5% per live births. PWB can exist alone or coexist with many other vascular malformations, such as Sturge-Weber syndrome, Parkes-Weber syndrome, and arteriovenous malformations (AVM). The potential treatment population will be over 3M in the U.S. alone.

The pulsed dye laser (PDL) or photodynamic therapy (PDT) is the treatment of choice for PWB. Unfortunately, complete removal of PWB occurs in less than 10% of patients. Second, about 20% of lesions have no response to laser treatment. Between 16.3% and 50% of patients experience re-darkening of their PWB as early as 5 years after multiple PDL treatments. The current laser-based modalities result in inadequate efficacies which urgently needs a solution for patients.

Accordingly, it is an object of the present disclosure to provide a repurposing of itraconazole and RA activators to improve the efficacy of laser treatment for PWB patients.

Citation or identification of any document in this application is not an admission that such a document is available as prior art to the present disclosure.

### SUMMARY

The above objectives are accomplished according to the present disclosure by providing in one instance a method for treatment of capillary vascular malformations. The method may include introducing at least one retinoic acid pathway activator to at least one cell exhibiting a Port Wine Birthmark, employing at least one pulse dye laser to engage the at least one cell exhibiting a Port Wine Birthmark, and employing the at least one pulse dye laser to engage the at least one cell exhibiting a Port Wine Birthmark reduces coloration of the at least one cell exhibiting the Port Wine Birthmark. Further, the at least one cell may be a stem cell or an endothelial cell. Still yet, the at least one cell may exhibit at least one laser resistant phenotype. Moreover, itraconazole may be introduced in addition to the at least one retinoic acid pathway activator. Furthermore, introduction of itraconazole may increase activation of at least one apoptotic pathway in the at least one cell. Still again, the method may include introducing all-trans RA, isotretinoin, tazarotene, liazorole or combinations of the above.

In a further instance, a method for improving laser-based capillary vascular malformation treatment is provided. The method may include introducing at least one retinoic acid pathway activator to at least one capillary vascular malformation, employing at least one pulse dye laser to engage the at least one capillary vascular malformation, and employing the at least one pulse dye laser to engage the at least one capillary vascular malformation reduces coloration of the at least one capillary vascular malformation. Further, the at least one capillary vascular malformation may comprise a Port Wine Birthmark. Still again, the at least one capillary vascular malformation may exhibit at least one laser resistant phenotype. Further again, itraconazole may be introduced in addition to the at least one retinoic acid pathway activator. Again yet still, introduction of itraconazole may increase activation at least one apoptotic pathway in the at least one capillary vascular malformation. Still again, the method may include introducing all-trans RA, isotretinoin, tazarotene, liazorole or combinations of the above.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows photographs of Port Wine Stain pathogenesis.

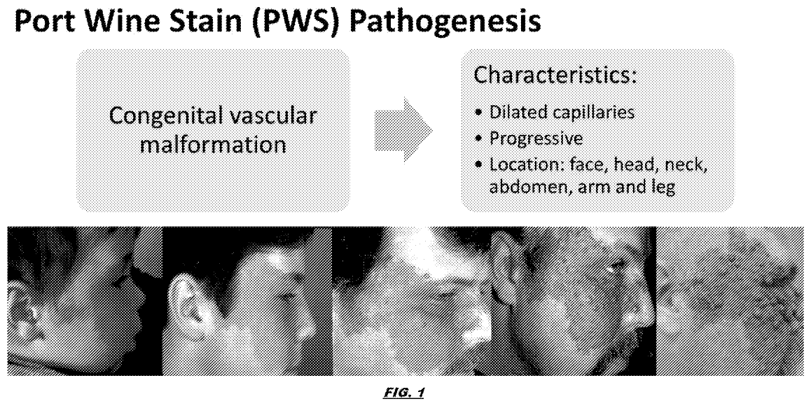

FIG. 2 shows an illustration of pulse dye laser treatment.

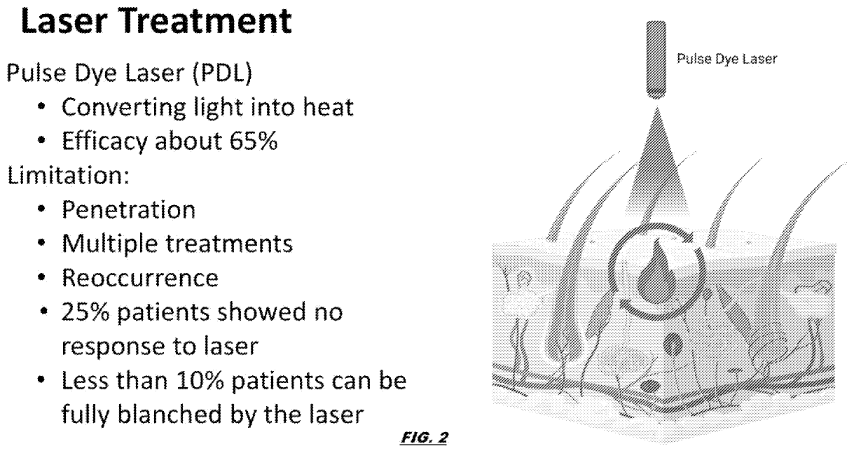

FIG. 3 shows an illustration of photodynamic treatment.

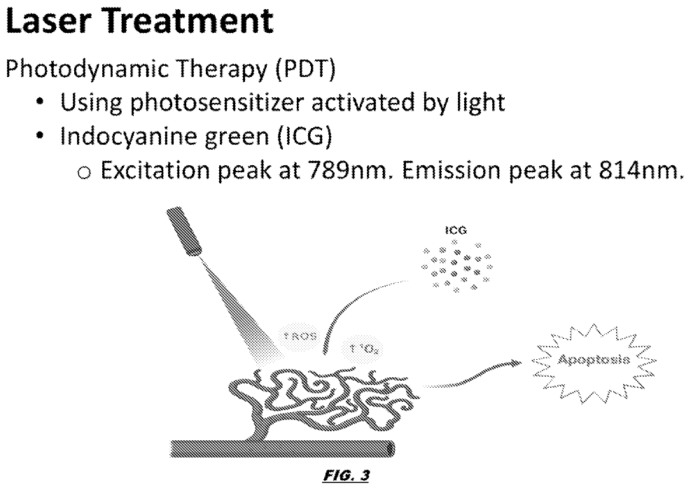

FIG. 4 shows an illustration of somatic cell reprogramming.

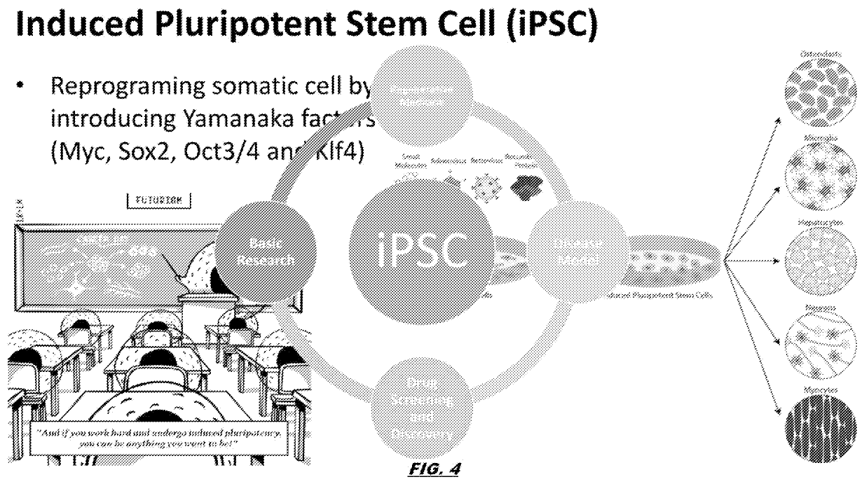

FIG. 5 shows images induced pluripotent stem cells from a donor without port wine stain and donors with port wine stain.

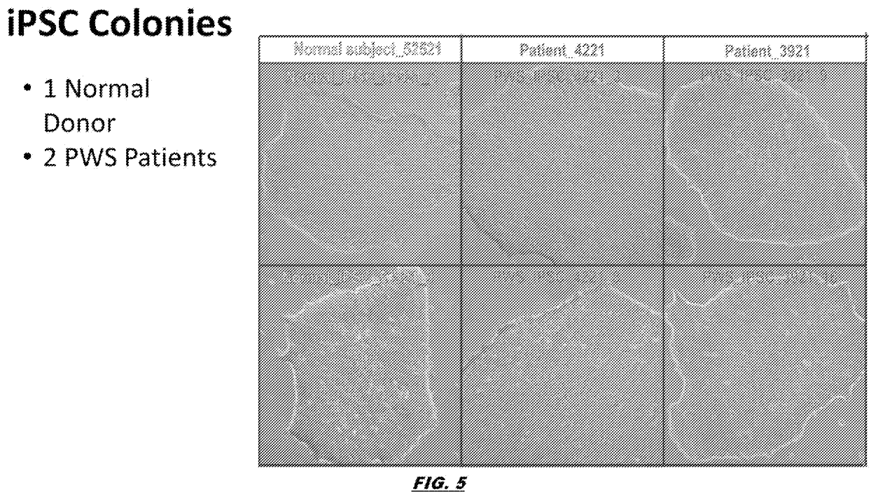

FIG. 6 shows markers iPSC colonies marked with stem cell markers.

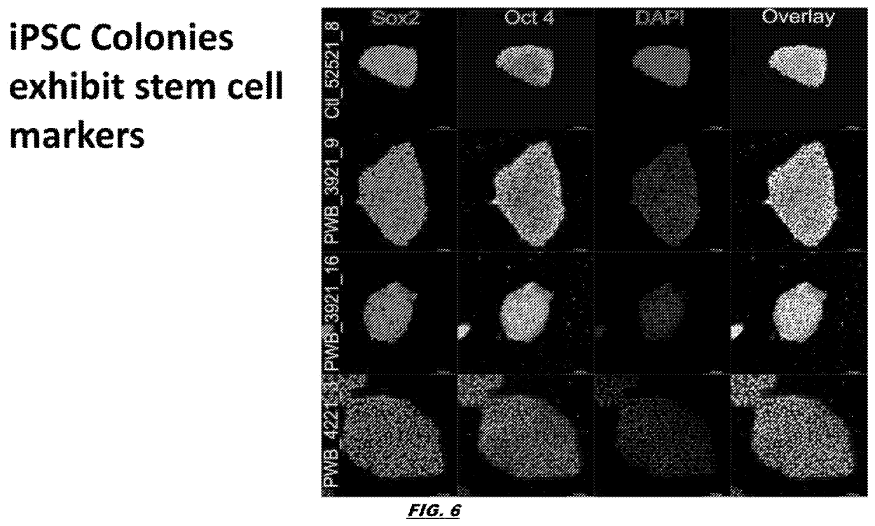

FIG. 7 shows a further image of iPSC colonies marked with stem cell markers.

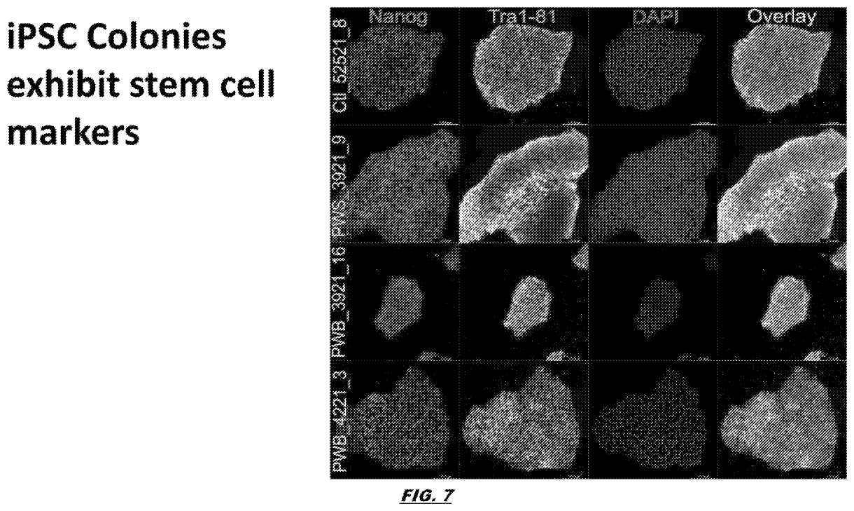

FIG. 8 shows an illustration of generating iPSC derived endothelial cells.

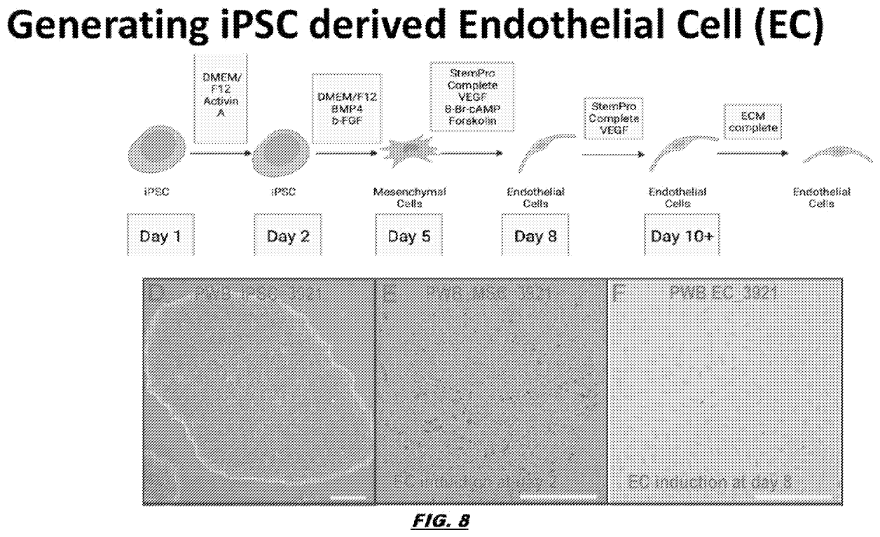

FIG. 9 shows iPSC derived endothelial cells showing endothelial cell markers.

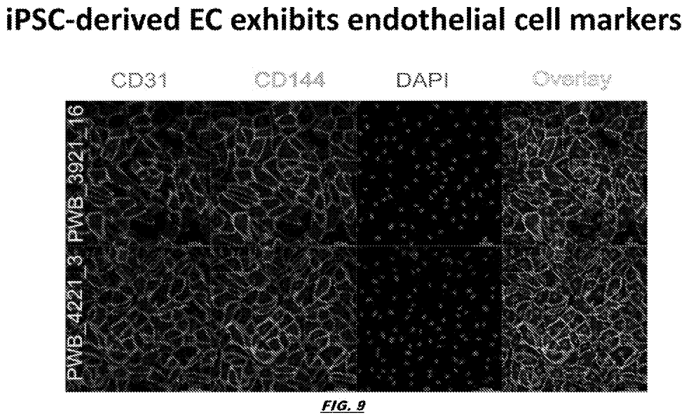

FIG. 10 shows images of iPSC response to PDT treatment.

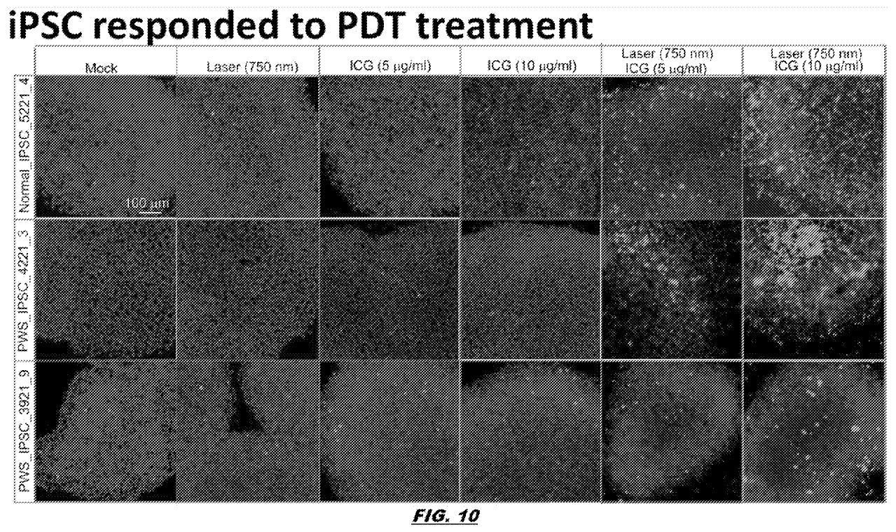

FIG. 11 shows an illustration that iPSC 3921 is laser resistant.

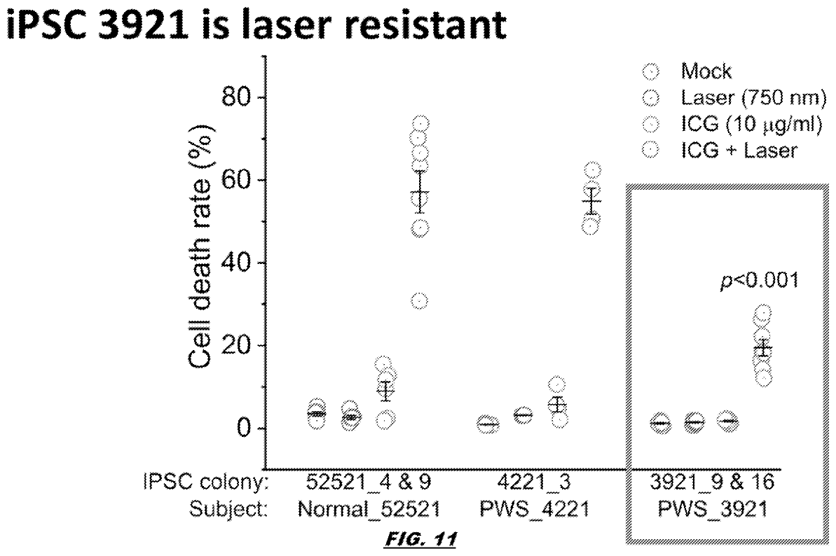

FIG. 12 shows preservation of an iPSC laser resistant phenotype.

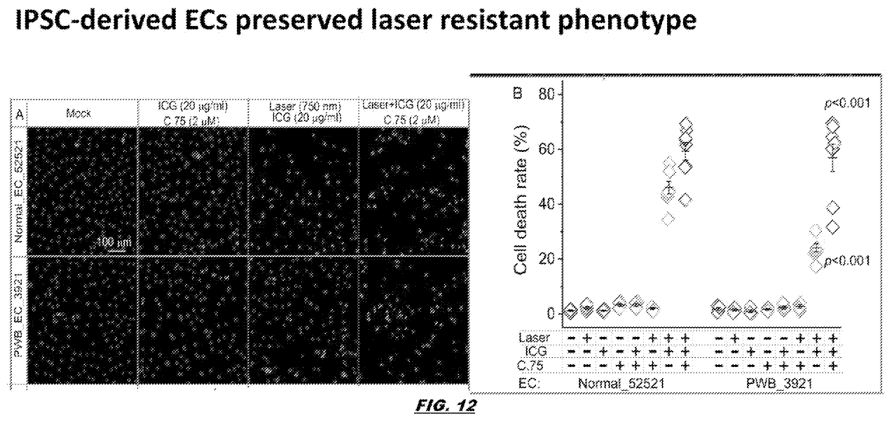

FIG. 13 shows a plot of PDT resistance vs. PDT-sensitive iPSC derived endothelial cells.

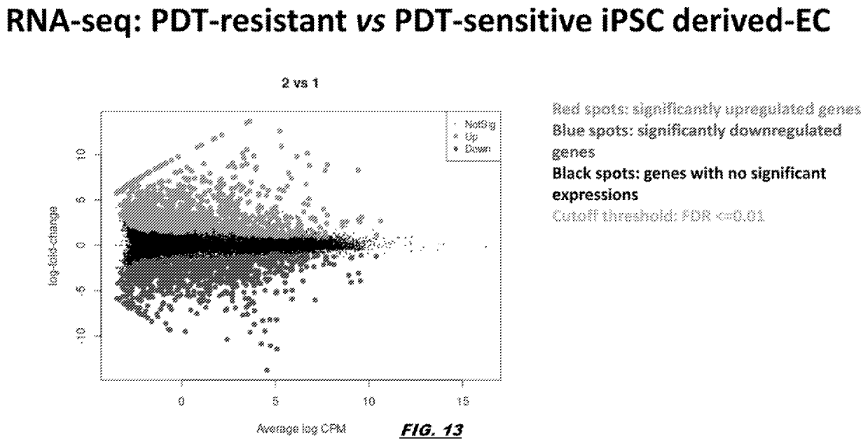

FIG. 14 shows an information table for KEGG pathway: EC_3921 Resistant vs. EC_52521 sensitive.

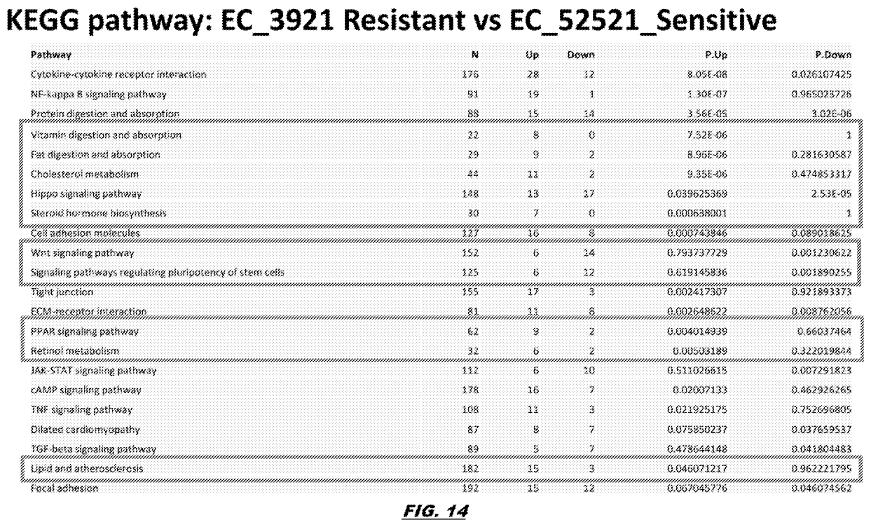

FIG. 15 shows an illustration of a multi-target drug schematic.

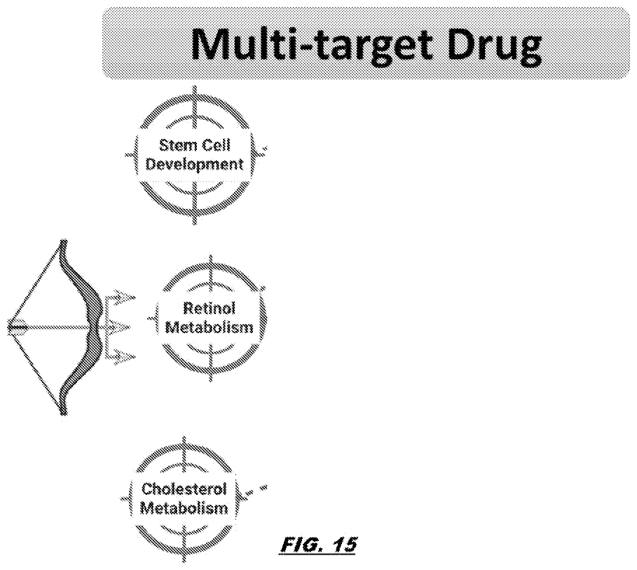

FIG. 16 shows the chemical structure for itraconazole and apoptosis induced by all-trans retinoic acid (ATRA).

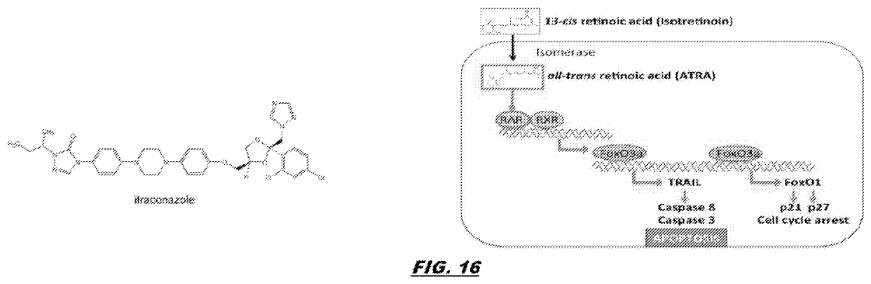

FIG. 17 shows photos of cell death showing itraconazole and other retinoid acid pathway modulators improve the efficacy of PDT.

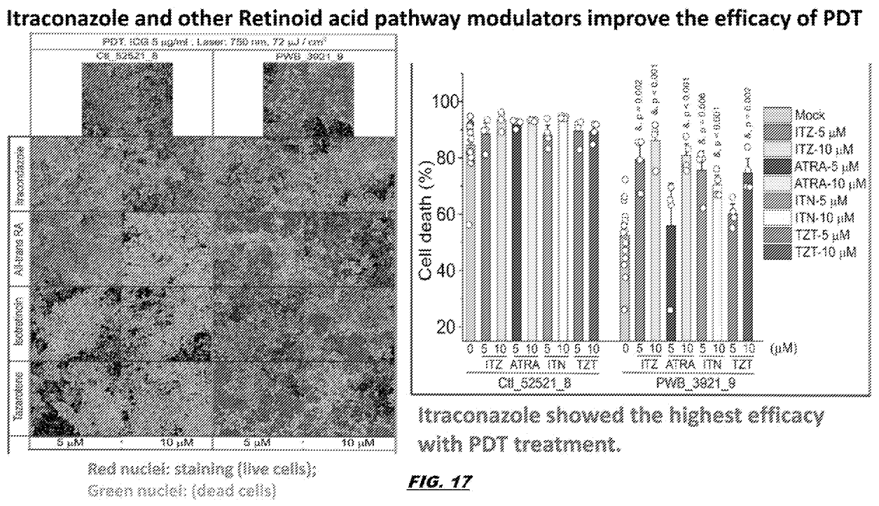

FIG. 18 shows apoptotic array photographs showing itraconazole increases apoptotic sensitivities to PDT in laser resistant iPSCs.

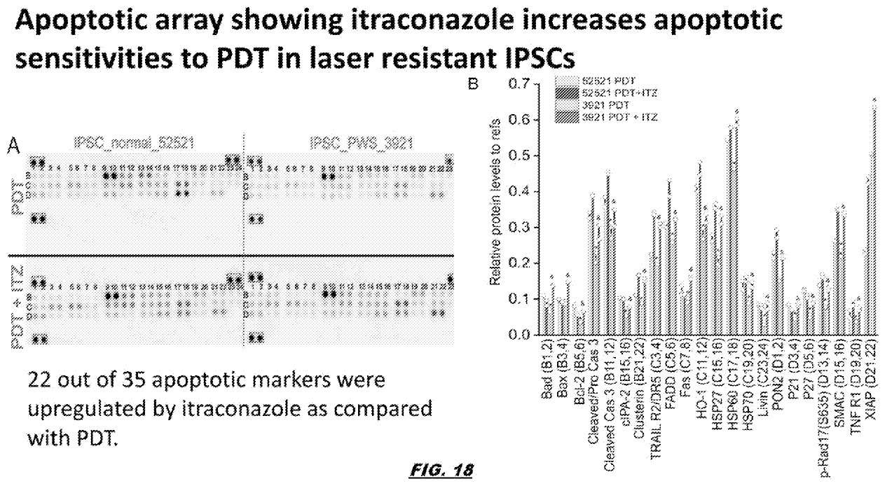

FIG. 19 shows kinase arrays demonstrating itraconazole reverses kinase phenotypes in response to PDT.

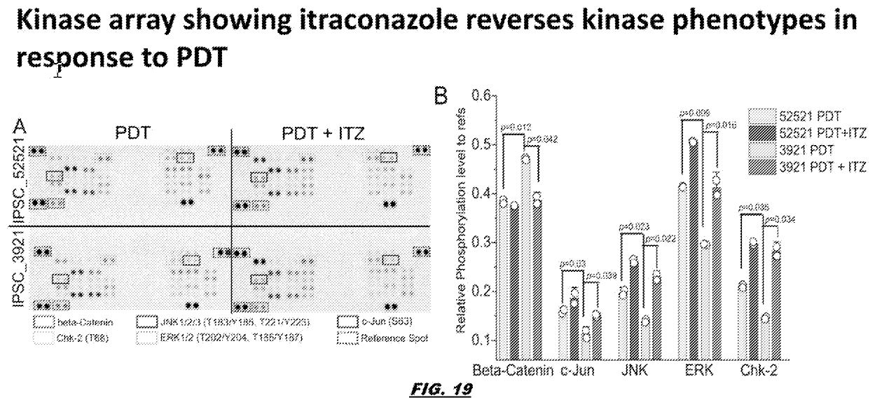

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “active agent” or “active ingredient” refers to a substance, compound, or molecule, which is biologically active or otherwise that induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition to the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “control” can refer to an alternative subject or sample used in an experiment for comparison purpose and included to minimize or distinguish the effect of variables other than an independent variable.

The term “optional” or “optionally” means that the subsequent described event, circumstance or substituent may or may not occur, and that the description includes instances where the event or circumstance occurs and instances where it does not.

As used herein, “dose,” “unit dose,” or “dosage” can refer to physically discrete units suitable for use in a subject, each unit containing a predetermined quantity of the active ingredient and/or a pharmaceutical formulation thereof calculated to produce the desired response or responses in association with its administration.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as port wine birthmarks or stains. The effect can be therapeutic in terms of a partial or complete cure of a disease, condition, symptom or adverse effect attributed to the disease, disorder, or condition. The term “treatment” as used herein covers any port wine birthmarks or stains, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “water-soluble”, generally means at least about 10 g of a substance is soluble in 1 L of water, i.e., at neutral pH, at 25° C.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

### Kits

Any of the compounds and/or formulations described herein can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agent(s)) contained in the kit are administered simultaneously, the combination kit can contain the active agent(s) in a single formulation, such as a pharmaceutical formulation, (e.g., a tablet, liquid preparation, dehydrated preparation, etc.) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds and/or formulations, safety information regarding the content of the compounds and formulations (e.g., pharmaceutical formulations), information regarding the dosages, indications for use, and/or recommended treatment regimen(s) for the compound(s) and/or pharmaceutical formulations contained therein. In some embodiments, the instructions can provide directions and protocols for administering the compounds and/or formulations described herein to a subject in need thereof. In some embodiments, the instructions can provide one or more embodiments of the methods for administration of retinol acid pathway modulators and CYP inhibitors and/or a pharmaceutical formulation thereof such as any of the methods described in greater detail elsewhere herein.

The current disclosure provides for repurposing of retinol acid pathway modulators and CYP inhibitors including FDA-approved and investigational drugs (such as itraconazole, all-trans RA, isotretinoin, tazarotene, liazorole, etc.) for treatment of capillary vascular malformations. These agents can improve the efficacy of energy-based modalities to ablate lesional vasculature and mitigate the re-occurrence of those lesional vasculature. The PDL or PDT is the treatment of choice for PWB. Pulsed dye laser (PDL) and photodynamic therapy (PDT) are both treatments for port wine stains (PWS), a type of birthmark. PDL is considered the standard treatment, but PDT has been shown to be an effective alternative.

### Pulsed Dye Laser (PDL)

PDL is a treatment that destroys capillary malformations using selective photothermolysis. It is considered the gold standard treatment for PWS, but its efficacy is limited.

### Photodynamic Therapy (PDT)

PDT is a vascular-targeted therapy that uses a photosensitizer to treat PWS.

Hematoporphyrin monomethyl ether (HMME) is a photosensitizer that's often used in PDT to treat PWS. PDT has been shown to be an effective and safe alternative to PDL.

Laser treatment for a port wine birthmark, also called a port wine stain, involves using a concentrated beam of light from a laser to target and destroy the abnormal blood vessels within the birthmark, causing it to fade and lighten in appearance; this is typically done with a pulsed dye laser, which is considered the gold standard for treating port wine stains, and usually requires multiple sessions to achieve optimal results.

Key Points about Laser Treatment for Port Wine Birthmarks:

### Targeted Blood Vessels:

The laser light is specifically absorbed by the red blood cells in the birthmark, causing them to break down and reducing the redness.

Multiple sessions needed. Most people require several treatment sessions spaced several weeks apart to see significant improvement. Early treatment is best. For best results, treatment is often recommended to start early in life when the birthmark is smaller and easier to treat.

Potential side effects may include temporary redness, swelling, bruising, and discomfort at the treatment site, which usually subsides within a few days.

Unfortunately, complete removal of PWB occurs in less than 10% of patients. Second, about 20% of lesions have no response to laser treatment. Between 16.3% and 50% of patients experience re-darkening of their PWB as early as 5 years after multiple PDL treatments. The current disclosure can improve the current efficacy of laser-based modalities.

The inventors recently generated patient-derived PWB Induced pluripotent stem cells (iPSCs). These cells show laser-resistant phenotypes as compared with normal controls. RNA-seq data showed that RA pathways are among dysregulated signalosomes in PWB iPSCs and ECs as compared with normal iPSCs and ECs. Induced pluripotent stem cells (iPSCs) can be differentiated into endothelial cells (ECs). ECs are cells that line blood and lymphatic vessels, and play a key role in the circulatory system. iPSC-derived ECs can be used in regenerative medicine and disease modeling. Patient-specific-iPSC-derived ECs can be derived from a patient's own cells, which can be useful for treating disease. Renewable source-iPSCs can proliferate indefinitely, providing a potentially unlimited source of ECs. Avoids patient-to-patient variability-iPSC-derived ECs can be a more consistent alternative to primary ECs, which can vary from patient to patient.

When the RA signaling activators were used, both PWB iPSCs and ECs showed higher responses to laser treatments. We found that itraconazole and RA activators improved laser response in PWB resistant iPSC; itraconazole showed the most efficacy. Itraconazole exacerbated activation of apoptotic pathways, cell death pathways that can be activated by internal or external signals, induced by PDT. This data demonstrates a repurposing of itraconazole and RA activators to improve the efficacy of laser treatment for PWB patients.

Photodynamic therapy (PDT) is effective in treating many types of skin conditions, including skin cancers, warts, acne, Port Wine Birthmarks, etc. PDT has high response rates and can clear lesions with excellent cosmetic results. Effectiveness. Superficial basal cell carcinoma (BCC): PDT has clearance rates of 80-97% for superficial BCC. Intraepidermal squamous carcinoma (IEC): PDT has a high response rate of 86.2% for IEC. Solar keratosis: PDT has a clearance rate of over 90% for solar keratosis. Microinvasive squamous cell carcinoma (SCC): PDT has a complete response rate of 84.2% after one session of ablative fractional laser-primed PDT (AFL-PDT). Survival: PDT has been shown to improve survival, with median survivals ranging from 330-493 days after diagnosis. PDT uses a photosensitizing agent that builds up in abnormal cells more than healthy cells. The agent is then activated with light, which kills the abnormal cells.

Some MAPK inhibitors such as axitinib have also been investigated by our group and other teams. However, the efficacy is unsatisfied. Also, the side effects are major concerns for pediatric patients. The costs are also high.

The itraconazole and other RA modulators have a good safety profile which will be applicable to pediatric patients. In addition, the cost of itraconazole is low, which won't cause a big economic burden to patients.

Further, itraconazole or retinoic acid (RA) activators could increase PDT efficacy in PWS-resistant vasculature by modulating apoptosis, angiogenesis, and stem cell development pathways.

The current disclosure provides that iPSC and their derived-EC preserved laser response phenotype in PWS. Itraconazole and RA activators improved laser response in PWS resistant iPSC. Itraconazole showed the most efficacy and exacerbated activation of apoptotic pathways induced by PDT.

These drugs can be used for treatments for congenital vascular malformations alone or in combination with other modalities (prior to, during and post) including but not limited to energy-based therapy, surgical therapy, etc. The inventors also believe it can be used for post-surgery for inhibition of abnormal blood vessel reoccurrence after laser or surgical treatments, etc.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A method for treatment of capillary vascular malformations comprising:
introducing at least one retinoic acid pathway activator to at least one cell exhibiting a Port Wine Birthmark;
employing at least one pulse dye laser to engage the at least one cell exhibiting a Port Wine Birthmark; and
wherein employing the at least one pulse dye laser to engage the at least one cell exhibiting a Port Wine Birthmark reduces coloration of the at least one cell exhibiting the Port Wine Birthmark.

2. The method for treatment of capillary vascular malformations of claim 1, wherein the at least one cell is a stem cell or an endothelial cell.

3. The method for treatment of capillary vascular malformations of claim 1, wherein the at least one cell exhibits at least one laser resistant phenotype.

4. The method for treatment of capillary vascular malformations of claim 1, wherein itraconazole is introduced in addition to the at least one retinoic acid pathway activator.

5. The method for treatment of capillary vascular malformations of claim 4, wherein introduction of itraconazole increases activation of at least one apoptotic pathway in the at least one cell.

6. The method for treatment of capillary vascular malformations of claim 1, further comprising introducing all-trans RA, isotretinoin, tazarotene, liazorole or combinations of the above.

7. A method for improving laser-based capillary vascular malformation treatment comprising:
introducing at least one retinoic acid pathway activator to at least one capillary vascular malformation;
employing at least one pulse dye laser to engage the at least one capillary vascular malformation; and
wherein employing the at least one pulse dye laser to engage the at least one capillary vascular malformation reduces coloration of the at least one capillary vascular malformation.

8. The method for improving laser-based vascular malformation treatment of claim 7, wherein the at least one capillary vascular malformation comprises a Port Wine Birthmark.

9. The method for improving laser-based vascular malformation treatment of claim 7, wherein the at least one capillary vascular malformation exhibits at least one laser resistant phenotype.

10. The method for improving laser-based vascular malformation treatment of claim 7, wherein itraconazole is introduced in addition to the at least one retinoic acid pathway activator.

11. The method for improving laser-based vascular malformation treatment of claim 10, wherein introduction of itraconazole increases activation at least one apoptotic pathway in the at least one capillary vascular malformation.

12. The method for improving laser-based vascular malformation treatment of claim 7, further comprising introducing all-trans RA, isotretinoin, tazarotene, liazorole or combinations of the above.
